(19) United States
(12) Patent Application Publication
(10) Pub. No.: US 2020/0098460 A1
(43) Pub. Date: Mar. 26, 2020

(54) SYSTEMS AND METHODS FOR IDENTIFYING INDIVIDUAL DOSES OF MEDICATION

(71) Applicant: EPIS Holding Company, LLC, Florence, SC (US)

(72) Inventors: Willard Dean Banks, Florence, SC (US); William Stuart Carroll, II, Darlington, SC (US); Roger Rolan Roff, Dillon, SC (US); John Bellamy, Chicago, IL (US)

(73) Assignee: EPIS Holding Company, LLC, Florence, SC (US)

(21) Appl. No.: 16/579,391

(22) Filed: Sep. 23, 2019

Related U.S. Application Data
(63) Continuation-in-part of application No. 16/526,432, filed on Jul. 30, 2019, now abandoned.
(60) Provisional application No. 62/736,808, filed on Sep. 26, 2018.

(51) Int. Cl. G16H 20/13; G16H 20/60; G16H 10/60; G06K 7/14

(52) U.S. Cl. CPC G16H 20/13; G06K 7/1417; G16H 10/60; G16H 20/60

(57) ABSTRACT

A system for uniquely identifying a medication unit such as a pill, a tablet, or a capsule includes a unique identifier located directly on the medication unit that distinguishes the medication unit from any medication unit. The system further includes a database that is hosted on a server device, the database being searchable to identify the medication unit and including the unique identifier and other information associated with the medication unit. The system further includes a scanner that scans the unique identifier to provide the unique identifier o a user device that communicates with the server device via a network to identify the medication unit.

### CROSS REFERENCES TO RELATED APPLICATIONS

This application is a continuation-in-part of U.S. patent application Ser. No. 16/526,432, filed Jul. 30, 2019, which claims priority to U.S. Provisional Patent Application No. 62/736,808, filed Sep. 26, 2018, each of which is incorporated herein by reference in its entirety.

### BACKGROUND OF THE INVENTION

There is a significant problem with the supply chain of prescription pharmaceuticals that includes illicit medication usage, counterfeiting, stealing, diverting, abusing, misusing and weaponizing prescription medications. The solution is to give each pill, capsule or tablet a unique identification mark, which makes it traceable from inception to end user. A technology to authenticate and validate a prescription medicine's ownership which establishes a certifiable method of traceability. Adding a unique identification mark on each dose of medication (e.g. pill, capsule, or tablet) will close the gap in the supply chain that occurs after the medication leaves the manufacturer by giving every dose of medication its own unique individual identifier, which allows each dose to be identified and tracked at every point along the supply chain through the end user. Additionally, a database can be created to store information associated with the dose of medication (i.e. medication, ingredients, dose, manufacturing information including lot and batch number, shipping information, pharmacy information, pharmacist filling the prescription, prescribing physician, patient information), and the information from this database can be used in many ways to limit and/or prevent many of the issues associated with medication today. Counterfeiting and the theft of medications had become such a significant problem in the United States that on Nov. 28, 2013 the Drug Quality and Security Act (H.R. 3204) was signed into law. The law calls for the U.S. Food and Drug Administration (FDA) to set standards for tracking and tracing pharmaceuticals, and phases in requirements for specified trading partners across the U.S. healthcare supply chain. Drug Quality and Security Act (DQSA) outlines steps to build an electronic, interoperable system to identify and trace certain prescription drugs as they are distributed in the United States. This law, once fully enacted, will enhance the FDA's ability to help protect the consumer from exposure to drugs that may be counterfeit, stolen, diverted, contaminated, or otherwise harmful. It requires manufacturers of pharmaceuticals sold in the U.S. to begin capturing and reporting product transaction histories, implement systems to report suspect or illegitimate products, and serialize the packaging for individual products (but not the doses of each product). The requirements, development of standards, and the system for product tracing will continue to be phased in until 2023 at which time all medications sold in the US will be tracked by barcode (or some other identifier) on a pallet of medication and on the bottle of medication. However, the DQSA tracking method does not identify an individual dose of a medication, such as a tablet, pill, or capsule.

Counterfeit medications, both abused and non-abused, are a problem in this country as well as the rest of the world, and it can be difficult to determine whether a medication is real or counterfeit. Counterfeit medications can be manufactured to look authentic and may be placed in containers that either are authentic (i.e. are in previously used containers of authentic medication) or appear to be authentic (i.e. old prescription bottles used to distribute authentic medication). The growing issue of counterfeit medications is a concern not only for the patient, but also for pharmacists and pharmaceutical companies. The World Health Organization (WHO) has estimated that 10% of global pharmaceutical commerce, or $21 billion worth, involves counterfeit drugs annually. Preventing counterfeit medicines from entering the supply chain and detecting counterfeits is often difficult, because many of these goods pass through a long and complicated distribution network, thereby creating opportunities for counterfeits to enter the legitimate supply chain. In addition to taking income from consumers and drug companies, counterfeit drugs also pose health hazards to patients, including death. Currently, there is no way to easily determine whether a single dose of medication is authentic or counterfeit. Identifying each individual dose of medication could help eliminate counterfeit medications from being distributed.

The Opioid Crisis is a huge problem. Prescription opioid medications play an integral part in the management of acute and chronic pain. Opioids have been regarded for millennia as among the most effective drugs for the treatment of pain. Their use in the management of acute severe pain and chronic pain related to advanced medical illness is considered the standard of care in most of the world. Opioid medications allow many people to be able to function in society and lead normal lives. However, there are a large number of individuals that abuse these types of medications. The abuse of prescription narcotic medications (e.g. opiates, opioids, and benzodiazepines) and other controlled prescription medications (collectively, “abused medications”) has reached epidemic proportions in America today and has been termed the “Opioid Epidemic”. Opioid addiction is one of the largest and most devastating epidemics in United States history. Every day, more than 116 people in the United States die after overdosing on opioids. The misuse of and addiction to opioids is a serious national crisis that affects public health as well as social and economic welfare. The Centers for Disease Control and Prevention estimates that economic cost of the opioid epidemic in 2016 was at $504 billion. The estimated cost of diversion alone being $72.5 billion per year and these numbers are continuing to grow. Many individuals are addicted to the abused medications, and, as a result, illicit activity arises from these addictions, including stealing of abused medications from individuals, pharmacies, and other sources; patients selling part or all of their prescribed medications; seeing multiple medical professionals for the same or fabricated condition who will provide a prescription for the opioids “doctor shopping”, etc. The result of the illicit activity is usually that the abused medications are possessed by people who do not have a legitimate medical need for the abused medications. Adding a unique individual pill, table, capsule marking will allow authorities, regulators, and providers to trace each pill to the identity of the end user. This unique identification mark would make it much more difficult for unauthorized persons to obtain medications and add accountability for those who divert medication. Currently when law enforcement locates an individual who should not have the abused medication, it is difficult if not impossible for law enforcement to determine what went wrong that led to the improper possession of the abused medications, especially when the abused medication is not in packaging (e.g. a prescription bottle, etc.) that identifies the source of the abused medication. This is because individual doses of the medication do not reflect this information. What is needed is a way to determine information associated with the abused medication and or weaponized medication or nutritional supplements to help uncover how an abused medication ended up in the wrong hands (and many other uses as further described herein). The information associated with the abused medications may be used by U.S. Defense Department, law enforcement, first responders, pharmacies, medical personnel, and others associated with this epidemic to limit access and/or prevent the unauthorized possession of abused or weaponized medications or nutritional supplements, to arrest guilty parties, etc. By making people accountable, this will limit unnecessary access to these medications. There is a significant amount of research and data to confirm that restricting access to drugs reduces abuse.

Every year, medications are recalled for various reasons. A drug recall occurs when medication (prescription or over-the-counter medicine) is removed from the market because it is found to be defective, potentially harmful, or otherwise too risky to remain available. Sometimes, the makers of the drug will discover a problem with their drug and voluntarily recall it. Other times, the FDA, or another governmental entity or oversight organization (the CDC, DEA, etc.) will request that the medicine be recalled after receiving reports of problems from the public. Yet other times, a portion of a medication is removed (i.e. one or more batches or lots of the medication). However, it can be difficult to determine whether an individual dose of medication is the subject of a recall.

Placing a scannable identifier on an individual dose of medication is known in the art. However, the identifiers used today are not unique to a single dose of medication but, instead, group several doses together (such as those created in the same batch, lot, etc.), which has many limitations. For example, this method of identification works best when a single patient receives medication bearing the same identification. This is problematic because, for instance, when a pharmacy does not have enough medication from one batch to fill a prescription, the pharmacy needs to either fill the prescription with doses bearing different identifiers or switch to medication from another batch in order to fill the prescription with medication bearing the same identification (which may result in discarding the remaining medication in the previous batch, which is wasteful). Not only is this method wasteful, it requires additional effort by the pharmacy or other provider filling the prescription. Moreover, a batch or lot of a particular medication may include thousands or even more than one hundred thousand doses. Batch-level identification does not provide the information needed to make dose-level determinations (i.e. proper patient, treating physician, pharmacy, correct dosing, or other information associated with a particular dose of medication) since so many doses of medication would share the same identification, it would be virtually impossible not to have a large number of individuals with medication that bear the same identifier and therefore no way to accurately authenticate ownership. In order to make dispensing the medication easier and less wasteful, to reduce illicit activity, to identify counterfeit medication introduced into the supply chain, to determine whether medication is the subject of a recall, to better fight the Opioid epidemic (and a host of other reasons), a unique medication identifier that identifies only one dose of medication, rather than all doses in a lot, batch, etc., is required.

Another benefit of the implementation of a unique medication identifier is improved patient safety as a result of providing medical professionals (e.g. physicians, nurses, pharmacists, etc.) with access to more information. Not only can information about a dose of medication be maintained in a database, the database may include, for instance, the identity of the patient who received the medication. This information may limit and/or prevent providing the patient with a new prescription that may be dangerous if taken with an existing prescription, prevent providing the wrong medication to a patient, providing the patient with an overlapping prescription that would violate state and federal laws, etc. Further, patient safety in hospitals, nursing homes, etc. may be improved by the use of a unique medication identifier. The unique medication identifier may eliminate dispensing errors that lead to adverse drug events, which are known to represent a major threat to patient safety despite widespread preventive programs and extensive education of hospital personnel. A unique medication identifier would reduce medication errors by electronically verifying the medication at the point of care to confirm that the right patient is receiving the right dose of the right drug at the right time.

Significantly, patient compliance is a significant problem with medications management. Research shows one of the leading causes of unnecessary deaths and one of the largest contributors to increased medical costs is medication non-adherence. Therefore, a need exists for technology to monitor and enforce patient compliance.

### SUMMARY OF THE INVENTION

According to an embodiment described herein, a medication that can be uniquely identified includes a unique medication identifier placed directly on the medication. Notably, improved compliance through the present invention helps improve overall health and safety. This unique medication identifier uniquely identifies the medication from all other medications. The medication corresponds to a single pill, capsule, or tablet. The unique medication identifier may uniquely identify the medication because no other medication has had the same unique medication identifier for a predefined period of time (such as 7 years, 10 years, etc.). The unique medication identifier may correspond to one or more of a 2D barcode, a 3D barcode, a string of alphanumeric characters, a microdot code, molecular bar coding or a taggant. The unique medication identifier may be mechanically affixed to the medication and/or placed on a label which is affixed to the medication. The medication may include a surface treatment, and the unique medication identifier may be located proximate the surface treatment. The medication may correspond to prescription medication, over-the-counter medication, a vitamin or a nutritional supplement.

According to another implementation described herein, a system for uniquely identifying a pill, tablet, or capsule includes a unique medication identifier located directly on the pill, tablet or capsule. The unique medication identifier distinguishes the pill, tablet or capsule from any other pill, tablet or capsule. The system further includes a database that is hosted on a server device, the database being searchable to identify the pill, tablet or capsule. The database includes the unique medication identifier and other information associated with the pill, tablet, or capsule on which the unique medication identifier is located. The system further includes a scanner that scans the unique medication identifier to provide the unique medication identifier, or a serialized version thereof, to a user device. The user device communicates with the server device via a network to query the database using the unique medication identifier, or a serialized version thereof, to obtain the information associated with the pill, tablet or capsule. When the unique medication identifier, or serialized version or thereof, is searched in the database, the database identifies the pill, tablet or capsule using the information associated with the pill, tablet or capsule. The scanner may be included in the user device. The scanner and the user device may be separate devices, and the scanner may provide the unique medication identifier, or serialized version thereof, to the user device via a network. The information associated with the pill, tablet or capsule may include the type of medication, dosage, lot number, batch number and any other production information desired. The user device or a second user device may communicate with the server device via the network to provide additional information associated with the pill, tablet or capsule to the database. The additional information associated with the pill, tablet or capsule may include the identity of the individual to whom the pill, tablet or capsule was prescribed; shipping information (supply chain) for the pill, tablet or capsule; the identity of the medical professional who prescribed the pill, tablet or capsule; and the identity of the pharmacist who filled the prescription for the pill, tablet or capsule. The user device or a second user device may communicate with the server device via the network to provide information associated with a patient to the database, the information associated with a patient may include allergy status of the patient, criminal history of the patient, or prescription history of the patient. The information associated with the pill, tablet or capsule may include the expiration date of the pill, tablet or capsule, medication that should be avoided when taking the pill, tablet or capsule, or the recall status of the pill, tablet or capsule. The information associated with the pill, tablet or capsule may include confirmation that the pill, tablet or capsule is not counterfeit or weaponized.

According to another implementation described herein, a server device that communicates with a user device to provide information associated with a pill, tablet or capsule to the user device includes one or more processors that execute instructions to receive from the user device a request for information associated with the pill, tablet or capsule. The request includes a unique identifier that uniquely identifies the pill, tablet or capsule in a database that is either stored on the server device or available to the server device via a network. The one or more processors identify the information associated with the pill, tablet or capsule in the database by matching the unique identifier from the user device to a second unique identifier in a profile for the pill, tablet or capsule. The one or more processors authorize the user device to access the information associated with the pill, tablet or capsule in the database or send a copy of the information associated with the pill, tablet or capsule to the user device. The pill, tablet or capsule corresponds to prescription medication, over-the-counter medication, a vitamin or a nutritional supplement. The information associated with the pill, tablet or capsule may identify the type of pill, tablet or capsule and the dosage of the pill, tablet or capsule. The one or more processors may receive from the user device or another user device second information associated with pill, tablet or capsule. The second information associated with pill, tablet or capsule may include recall status of the pill, tablet or capsule. The one or more processors may receive from the user device or another device information associated with one or more individuals. The information associated with one or more individuals may include criminal history or prohibited medications for the individuals.

### BRIEF DESCRIPTION OF THE DRAWINGS

FIG. 1 illustrates a non-limiting example embodiment of a medication that may be used in connection with the systems and/or methods described herein.

FIG. 2 an example environment in which the systems and/or methods described herein may be implemented.

FIG. 3 is a flow chart of an example process in which information is shared via a database according to an implementation described herein.

FIG. 4 is a schematic illustrating the process for marking a medication unit according to one embodiment of the present invention.

FIG. 5 is a schematic diagram of a system of the present invention.

### DETAILED DESCRIPTION

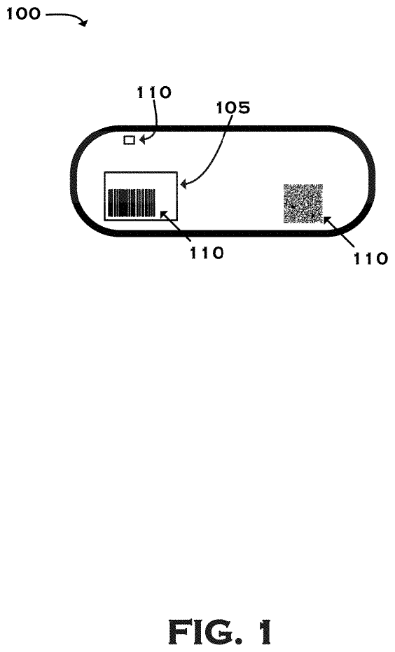

FIGS. 1-5 are attached hereto and incorporated herein by reference. The following detailed description refers to the accompanying FIGS. 1-5. The same reference numbers in different figures may identify the same or similar elements.

The systems, methods, technologies and/or techniques (hereinafter, the “systems and methods”), described herein, may include a medication unit (a dose of medication) that includes a unique medication unit identifier, also referred to as a unique medication identifier herein, which may be interrogated by a scanning device (e.g. a scanner, a barcode reader, an electro-optic device, etc.) to provide information associated with the medication. The unique medication identifier may uniquely identify a single dose of medication (individual pill, tablet, capsule, or other form of single dose medication, including liquid single dose medications) so that it can be tracked, authenticated, distinguished from other doses of medication, and otherwise enhance the administration of medication using the systems and methods described herein. In addition to a scannable unique medication identifier, the systems and methods provided herein may be implemented with unique medication identifiers that can be read without a scanner, such as by the human eye. However, scannable identifiers are referred to herein because these types of identifiers are associated with more possible unique codes than what may traditionally be read by humans without assistance.

In one embodiment, the individual identifier or unique medication identifier includes a serial number or has a serial number component. In a preferred embodiment, the unique medication identifier includes a combination of at least one of a serial number component or a unique symbol. In alternative embodiments, taggants and microdots are used for identifying individual medication units wherein the medication unit includes a single dose of a medication. The taggants and microdots do not require incorporation of alphanumeric characters or symbols; however, alphanumeric characters or symbols are incorporated into the taggants and/or microdots in one embodiment. Note that prior art taggants and microdots are not acceptable for human consumption. However, the embodiments of the present invention incorporate taggants and/or microdots that are acceptable for human consumption. In the present invention detailed description, the term “individual identifier” is intended to include this technology in forms that are acceptable for human consumption. In yet another embodiment of the present invention, an individual serial number is provided for each pill, capsule, tablet, medication unit or dose equivalent and affixed thereto or inextricably linked or connected thereto.

Each form factor for medication delivery, including but not limited to at least one pill, capsule, tablet, medication unit, dosage or dose equivalent of medication is associated with a unique medication identifier. The unique medication identifier is affixed to each individual dose of medication, and upon interrogation by a user device, may provide information associated with the individual dose of medication. The unique medication identifier may vary depending upon the type of medication and/or dosage unit (e.g. solid tablet, liquid-filled capsule, powder-filled capsule, etc.) but may generally include multi-digit codes, strings of alphanumeric characters, molecular barcodes, symbols (e.g. barcodes, 2-D barcodes, 3-D barcodes, etc.) or other types of marking and/or machine readable indicia that may be placed on a medication and interrogated, such as by a scanning device, to identify the medication. The unique medication identifier is operable to be converted into a data matrix code, quick read code, etc. for the individual identification of a pill, capsule, or tablet so that it can be tracked and traced from inception to end user and identify counterfeiting or weaponizing. The unique identifier is also operable to uniquely identify a single dose of the medication such that two doses of medication are operable to be distinguished from each other based upon having different identifiers (i.e. no two medications bear the same unique identifier at any time). This is possible because many types of codes available today have literally trillions of different available codes, each of which could be associated with a single dose, and more than one type of code may be utilized in connection with the systems and methods described herein. As a result, every single prescription in pill, tablet, capsule, etc. form may bear at least one unique identifier that is not shared by any other dose or unit of medication (or, at least, no other doses during the average life of the medication). Alternatively, the time period for reusing a unique identifier for a medication is based on a predetermined period of time, such as 10 years, 15 years, 20 years, etc.

The unique medication identifier is also operable to include or be solely in the form of a taggant. In one embodiment, a taggant is a chemical marker or a physical marker attached to or integrated with a pill, capsule, or tablet or incorporated into a liquid via dissolution or suspension to allow various forms of unique individual identification. By way of example, a physical marker taggant includes microscopic particles of varying color sequences or a conglomeration thereof that can be read by a reader such as an optical reader. A physical marker also includes line patterns or types such as rods, arches, loops, or whorls within a pattern area similar to those found in the human fingerprint according to one embodiment of the present invention.

In another embodiment, the unique medication identifier is operable to include or be solely in the form of a microdot. By way of example and not limitation, a microdot includes the application of small particles inscribed or etched with a unique identification code that includes one or more numbers, letters, symbols, colors or color patterns etc. or any combination thereof that is operable to be read by a reader. A microdot is also operable to act as a unique identifier by emitting a radiofrequency, wavelength, electromagnetic charge, or other type of signal that is unique and can be detected by a reader or reading device. The reader device of the present invention is operable to detect a signal using a diffraction method which allows the marked medication to be identified while inside a container such as a bottle.

In another embodiment, the unique medication identifier is operable to include or be solely in the form of a molecular barcode. By way of example and not limitation, a molecular barcode refers to a unique nucleic acid sequence, such as a deoxyribonucleic acid (DNA) sequence, or more generally, a unique sequence or arrangement of a group of molecules. While DNA barcoding typically involves 200 to 600 base pairs (or more), DNA mini-barcoding is also operable to uniquely identify a medication according to the present invention. The molecular barcode is also operable to include other particles or molecules, such as nanoparticles, to create a unique identifier. The molecular barcode provides for tracking the medication through space and time through the use of common genetic techniques known in the art such as Polymerase Chain Reaction (PCR).

The unique medication identifier may be placed on, imprinted onto, affixed to, and/or attached to any location of the medication that may be interrogated by or input to a scanning device or any other mechanism for receiving or perceiving the unique medication identifier. Ideally, the location of the unique medication identifier is placed on the medication in a way that facilitates scanning the medication, such as by facilitating alignment in a hopper or other device that allows multiple doses of the medication to be quickly scanned, such as at the manufacturer or a pharmacy filling a prescription. The location of the unique medication identifier may be selected to avoid affecting the medication. For example, powder filled capsules may be marked where portions of the capsule overlap to avoid weakening the capsule (e.g. by etching used to create the unique medication identifier, etc.) by placing the unique medication identifier on an area of increased capsule thickness. Similarly, gel caps may be marked in a sealing area, where the capsule portion is thicker, or in another area where the unique identifier does not affect the medication by, for instance, weakening the capsule or making a thin area on the capsule that causes the capsule to deliver the medication quicker than designed because the thin area dissolves quicker than the rest of the capsule. Multiple copies of the unique medication identifier can be placed onto an individual medication, especially medication designed to be split, so that the medication can still be identified after being split. The unique medication identifier may be applied in a variety of ways, including, but not limited to, laser etching, printing, embossing, debossing, engraving, press marking, roll marking, dot open peen marking, stylus marking, stamps, labels or stickers (preferably bio-absorbable) overlays, scribes, microdot, taggant technology, micro-taggant technology, etc. Therefore, a variety of equipment may be used to apply the unique medication identifier to the medication.

The unique medication identifier may be placed on the medication anywhere along the supply chain for the medication. Ideally, the unique identifier is placed on the medication by the manufacturer. In one embodiment, the unique identifier is assigned from a database on a server computer in communication with a manufacturer computer connected to a printer or other device operable to print, tag, or otherwise mark a medication with a unique identifier to a computer attached to the printer at the manufacture. Once a manufacturer is ready to distribute the medication, the manufacturer computer contacts the database on the server computer and the server computer utilizes one or more algorithms to assign randomized identifier which is non-sequential relative to other unique medication unit identifiers for medication units within a prescription and is non-duplicated compared to all other unique medication unit identifiers for that medication and sends these characters to the printer at the manufacturer. Upon assignment of the unique medication identifier, the manufacturer computer sends data about the medication to the server computer, including but not limited to, primary data such as the name of the medication, a lot, a batch, a date, a location, etc., which is stored in the database on the server computer.

At that point, a database may be populated such that all of the information that is presently known about the medication may be associated with the unique identifier for the medication. For instance, information that is known at the time of manufacture—such as the type of medication, manufacturer identity, date of manufacture, lot number, ingredients, supplier of ingredients, etc.—may be populated in the database by the manufacturer such that the unique medication identifier is associated with the information. Proprietary information including trade secrets, such as the formula of a medication, are preferably not included in the database. Information associated with medication that is not known until after manufacture—such as distribution channels, the individual to whom the prescription was written, the identity of the prescribing medical professional, the pharmacy at which the prescription was filled (and identity of the pharmacist), the date the prescription was filled, the number of doses in the prescription, etc.—may be provided to the database by the pharmacist, someone else in the supply chain, or another individual associated with the transaction for the medication. Manufacturers, distributors, supply chain managers, medical professionals, pharmacists, law enforcement, first responders, and others having an interest in the medication may access the database, such as via a network, to obtain the information associated with medication, to add additional information associated with the medication, to make decisions based on the information associated with the medication, etc. The database may be stored on one or more server devices connected to the network and accessed through a digital application.

To access the information associated with medication, a scanning device may interrogate (e.g. scan, read, photograph, etc.) the medication to read the unique medication identifier. Reading the unique medication identifier may include serializing the unique medication identifier to convert it into, for instance, an alpha-numeric string that represents the unique medication identifier in the database. The scanner may connect to the network to provide the unique medication identifier to the database to query the information associated with medication. Additionally, or alternatively, the scanner may communicate with (e.g. via a wired or wireless connection, local area network, the internet, etc.) another device (e.g. a desktop computer, a laptop computer, a tablet, a smart phone, etc.) to provide the unique medication identifier to the user device. The user device is operable to connect to the network to provide the unique medication identifier to the database to query information associated with the medication using the unique medication identifier. The at least one server associated with the database is operable to execute instructions to compare the unique medication identifier provided by the scanning device (or another device) to other unique medication identifiers in the database until the matching unique medication identifier is located in the database. The user device may be equipped with biometric sensors that are used in determining and verifying the identity of the device user. Smart phones on the market are equipped with fingerprint sensors, iris sensors, facial sensors, etc. which can all be implemented. Biometric information or data includes a fingerprint, one or more facial features operable to be recognized via facial recognition software, one or more features of an iris, one or more features of one or more veins including finger veins (e.g. finger vein recognition), palm veins (e.g. palm vein recognition), or any other unique identifying feature of a user. The database may link biometric information of a user interrogating a medication with unique medication identifier information when querying for enhanced compliance; wherein the unique medication unit identifier is associated with at least one biometric identifier operable to identify a user to whom the medication unit is prescribed. For example, when a patient is going to consume a medication, the patient will scan the medication's unique medication identifier through the mobile computing device application and at the same time biometric sensors on the phone will confirm the identity of the patient. One or more biometric sensors of the user device are operable to confirm the identity of the user in real-time or near real-time at the pharmacy and send a verification of the user's identity to a device at the pharmacy to verify the user's identity via an encrypted wireless communication using Near Field Communication (NFC), radio frequency (RF), Bluetooth, etc. Alternatively, the pharmacy includes a separate device which is operable to verify a user using facial recognition, fingerprint recognition, iris recognition, vein recognition, etc. Preferably, the user's biometric data is compared to biometric data stored in a secure database which the pharmacy is operable to access to verify the user's identity. The current method for verification of identity when picking up controlled substances from the pharmacy is by driver's license; however, many fake identifications are used in the process. A biometric identifier is much more difficult to fake and can provide accurate user identification information to the database. The information associated with the medication (or a subset thereof) that corresponds to the matching unique medication identifier and biometric information may be provided in response to the query from the scanning device (or other device). Additionally, or alternatively, the party scanning the unique medication identifier may update the database to provide additional information associated with the medication.

The database is populated with user and associated user information as well as drug and associated drug information. User profiles are made by the users through the user device application using their social security number for unique identification in the United States or the equivalent identifier in other countries. If a user is a medical professional, law enforcement agent, or a first responder, they can input their government registration and identification information for their profession into their profile, which is verified through a government database or another authoritative source, in order to differentiate their profiles from end-users' profiles. Biometric information is input and uploaded by the user through the user device and server application to their profile, but must be done at an authorized location with at least one authorized personnel. Doctors, pharmacists, other authorized medical professionals, and authorized officials may add information to the user profile pertaining to the user's public and private health records, identification information, as well as legal records associated with health. Doctors can input health information related to their patient that can assist pharmacists with their drug interaction protocols such as which other medications the patient is prescribed as well as their vital health parameters.

Uploading of biometric information must be performed at an authorized location with one or more authorized personnel to ensure that the biometric information is indeed the user's biometric information. Locations authorized to assist users in uploading their biometric information include doctor's offices, pharmacies, hospitals, and police stations. Authorized personnel include any persons working for one of the authorized locations. The authorized personnel provides limited access rights for the user to upload their biometric information to the user's profile at that instance only, or uploads the biometric information to the user's profile for the user. A user must use this biometric identification at the pharmacy when the user is picking up a prescription to ensure that the user is in fact the patient prescribed that drug, a legal guardian of the patient prescribed the drug, or a party with a power of attorney for the patient. All users must have biometric information associated with their profile uploaded to the database. First responders, medical professionals, and law enforcement will be able to access medication information for any drug using their biometric identification while interrogating a drug through a user device and application. Manufacturers will be able to access all drug information without any private patient information, in cooperation with HIPAA, using their biometric identification. All other users will only be able to access the information associated with their profile and any other profile to which they have been granted access to such as for a legal guardian or power of attorney.

Drug and associated drug information can be populated into the database by any user, however, with specific restrictions. The drug manufacturer is responsible for input of all information associated with the drug's manufacture such as date and time, type of medication, location of manufacture, batch number, lot number, ingredient suppliers, etc. Once manufacturers input the manufacturing information into the database and are assigned a unique medication identifier, the information associated with affixing the unique medication identifier is stored into the database such as which unique medication identifier went to which drug and when and where it was affixed to the drug, both physically on the drug and in space. Manufacturers and supply chain tracking managers are also responsible for inputting information about which container each individual dose of medication is placed into as well as which pallet the bottle is on. Each container is given a unique container identifier, and wherein the unique medication unit identifier is linked to the unique container identifier. Every pallet is given a unique pallet identifier wherein the unique pallet identifier is linked to the unique medication unit identifier and unique container identifier. When a dispensary receives a shipment of medication, the receiving official user, either a pharmacist or other authorized dispenser, uses their scanning device coupled with biometric sensors to confirm the receipt of the shipment as well as their identity to the server. Official users at the dispensary are also responsible for assigning the unique individual doses of medication to a patient user so that the patient's biometric information stored in the database can be used during pick up. Pharmacists as well as drug manufacturers and government agencies can input information pertaining to a drug or batch lot. For example, if a certain lot of drugs is found to be contaminated, an official user can issue a recall statement through the application. A patient or authorized user such as a caregiver or party with power of attorney can input drug information into the database such as when the patient scanned and consumed a dose of medication, side effects from the medication, and any other important drug-related information. Patient compliance to a medical treatment can be confirmed by medical professionals in this manner.

The unique container identifier is a randomized identifier which is non-sequential relative to other unique container identifiers and is non-duplicated compared to all other unique container identifiers. The unique container identifier includes multi-digit codes, strings of alphanumeric characters, molecular barcodes, symbols (e.g. barcodes, 2-D barcodes, 3-D barcodes, etc.) or other types of marking and/or machine readable indicia that may be placed on a container and interrogated by a scanning device. The unique container identifier is operable to be converted into a data matrix code, quick read code, etc. for the identification of all unique medication unit identifiers contained within, so that it can be tracked and traced from inception to end user and identify counterfeiting or weaponizing.

The unique pallet identifier is a randomized identifier which is non-sequential relative to other unique pallet identifiers and is non-duplicated compared to all other unique pallet identifiers. The unique pallet identifier includes multi-digit codes, strings of alphanumeric characters, molecular barcodes, symbols (e.g. barcodes, 2-D barcodes, 3-D barcodes, etc.) or other types of marking and/or machine readable indicia that may be placed on a pallet and interrogated by a scanning device. The unique pallet identifier is operable to be converted into a data matrix code, quick read code, etc. for the identification of all unique container identifiers contained within and the associated unique medication unit identifiers, so that it can be tracked and traced from inception to end user and identify counterfeiting or weaponizing.

Each user has a unique profile that can be accessed through a username and password and/or biometric information associated with a user. End-users may make inputs into the database through the user device and application. These inputs include recording when a medication is taken by scanning the unique medication identifier before consumption, recording side effects of the medication, or any other relevant drug information. For instance, if an end-user has an allergic reaction after consuming a medication, the user may input that information into the database through the user device and application. This can be useful for discovering issues with medications at scale and issuing a recall since patterns among medications can be linked to specific events or details much quicker.

Information associated with the medication on which the unique medication identifier is placed may be stored in a database that is accessible by law enforcement, manufacturers, others in the medication supply chain (medical professionals, pharmacies, regulatory agencies, etc.), the patient, and others associated with the medication. Because the identifier is unique, detailed information on the medication may be stored in the database, which can be interrogated to address many issues present in this market today. The information associated with the medication may include, but is not limited to: the type of medication (i.e. the ingredients, the brand name, etc.), the identity of the manufacturer of the medication, the location of manufacture, the date of manufacture, the date of expiration, additional manufacturing information (e.g. lot number, batch number, suppliers of ingredients, etc.), a picture of the medication, the identity of the pharmacy that provided the medication to a user, how the medication was distributed, the identity of the pharmacist who filled the prescription, the identity of the authorized prescription holder and/or guardian thereof, the prescribing medical professional, the date of the prescription, the date on which the prescription was filled, the extent to which the medication is subject to governmental control as a narcotic or other restricted pharmaceutical product, the authenticity of the medication, etc. The database may be interrogated to limit and/or prevent unauthorized access to abused medications as further described herein (e.g. by making arrests, determining the source of unauthorized distribution, etc.). Further, the unique identifier may limit and/or prevent the introduction and/or use of counterfeit or weaponized medication.

Counterfeit medications entering the supply chain would be identifiable. Since each pill capsule or tablet has its own unique identifier for at least the average life of the medication, once that identifier or serial number has been used, if it shows up again it would indicate counterfeit medication. The serialized unique medication identifiers are non-sequential, randomized, and assigned based off a non-duplication algorithm, and therefore will identify counterfeit medication entering the supply chain if a duplication arises or a unique medication identifier is associated with the wrong medication. For instance, the unique medication identifier could be scanned before an individual medication goes into the bottle and could be associated with the barcode on the bottle and the barcode on the pallet, so that it would be known where specific pills are in the supply chain. In this manner, every pill could be identified throughout the complete supply chain at any point in time. Even if the unique medication identifier were not attached to the barcode on the bottle or pallet, it could be determined if the pill was lost before reaching the dispensary or after, and if after, who it belongs too. If a pill has a unique medication identifier associated with it that has not been assigned by the database, the database would know that it has not been assigned and flag it as counterfeit or weaponized. If the unique medication identifier has been assigned, but does not match the type of medication then it is counterfeit.

This system and method can be used to track the source of deviations during the transport of the medication. In the case of arrests where medications with controlled scheduling are discovered, officials can use a scanning device to query the database for information associated with that dose of medication such as who prescribed it, which pharmacy and pharmacist dispensed it, who the patient is, and any other legally attainable information. This can be especially useful when seeking to identify if a doctor may be overprescribing a medication or if a patient is abusing the system by illegally distributing the medication. If the arrested individual's biometric information does not match what is stored on the database and/or the query returns that the medication has been marked stolen by an authorized user, then it can be determined that they obtained the medication illegally and the consequences can be sufficiently determined.

The unique medication identification mark on each pill is preferably scanned by a mobile computing device running an application before each pill is consumed. The device including the reader application is operable to send the patient a reminder relating to a next dosage date or a next dosage time for another medication unit within the single prescription, time for a prescription refill, and at other significant times relating to the patient and the medication. A reminder to take the next dose is particularly useful in cases where there is a tapering or titration of dose and compliance is crucial. Other notifications associated with the medication may be sent to the user device such as recall or product tampering information.

The information associated with medication may be used in a variety of ways, and the database may be used for uses beyond the particular medication being scanned. By way of example and not limitation, first responders, the U.S. Defense Department, law enforcement, a pharmacy, a treating physician, or other medical professional may interrogate the database for a prescription history of a particular patient. In response, the one or more servers associated with the database may execute instructions to determine the number of prescriptions associated with the patient, the medications provided to the patient, the dates of the prescriptions, a picture of the patient, biometric information of a patient, the prescribing medical professionals associated with the patient, etc. In this way, the database may be used to determine whether a patient has been “doctor shopping” or is being overprescribed certain types of medications, like opioids. The servers may provide this information in real time in response to a query, which may lead to certain decisions (e.g. refuse to fill the medication, place a “flag” on the individual, call a prescribing medical professional, notify law enforcement or other appropriate authority such as the United States Department of Defense, make an arrest, notify the patient of a dangerous combination of prescribed medications, etc.) based upon the information. This may limit and/or prevent an individual from obtaining a prescription that the individual should not obtain (e.g. because the individual has outstanding prescriptions on abused medications, is associated with professionals who over prescribe abused medications, etc.), may help identify medical professionals who overprescribe controlled medications, etc.

Alternative uses of the systems and methods described herein may include interrogation of the database by law enforcement to determine, for instance, whether an individual should possess a medication and, if not, to whom the medication was prescribed so that the source of unlawful distribution may be identified, the identities of physicians who prescribe abused medications is discovered, the identities of individuals who have multiple prescriptions for abused medications is discovered, etc. Further, law enforcement may determine that the medication was stolen from the dispensary or earlier point in the distribution channel if no information was uploaded to the database by dispensary. Other uses may include, but are not limited to, the identification of patients and/or pharmacies who possess expired medications, medications recalled by the manufacturer, FDA, CDC, etc. Alternatively, the servers may include permissions that allow a reduced amount of information associated with medication to be provided in response to a query, such as whether the medication is expired, subject to a recall, or weaponized, or simply the medication type and dosage. This information associated with medication may be accessed by a first group of individuals (such as an unrestricted group), whereas certain other of the information associated with medication may be restricted to a second group of individuals (i.e. restricted to only those individuals who have a legitimate need for the information, such as law enforcement). Restricting access to certain types of information associated with medication may be needed to comply with applicable law (e.g. to avoid breaking laws associated with protecting the privacy of medical information, like HIPAA, etc.), and/or other justifiable reasons to restrict access to some or all of the information associated with medication.

Reduced medication errors in hospitals, nursing homes, assisted living facilities, and other locations providing medical treatment may result from the systems and methods provided herein. For example, a nurse or other treating professional may scan the medication before providing it to a patient, and, in response, may receive information associated with medication that indicates to the nurse or other treating professional that the patient is not the proper subject for the medication, that the patient cannot take the medication (e.g. due to another medication, because not enough time has passed since the last dose, etc.). Further, the unique medication identifier may help eliminate counterfeit medications, from entering the supply chain by allowing the counterfeit medication to be easily identified with a scanning device (i.e. because it does not match the medication in the database, because there is more than one dose of medication with the same unique identifier, etc.). Further still, the database may be accessed by the manufacturer, or another party (e.g. FDA, CDC, etc.) to associate the unique medication identifier with a recall. When the unique medication identifier is scanned at the point of use (e.g., by a medical professional, by the patient, etc.), the database may be interrogated to determine whether the medication is the subject of a recall. These uses, and the many others anticipated by the systems and methods described herein, may help address the current crisis associated with abused medications in America and throughout the world all while improving public health and safety.

FIG. 1 illustrates a non-limiting example embodiment of a medication that may be used in connection with the systems and/or methods described herein. As shown in FIG. 1, the medication 100 may include one or more unique medication identifiers 110. The medication shown in FIG. 1 is provided for explanatory purposes only, and the disclosure herein is not intended to be limited to what is reflected in FIG. 1. There may be additional varieties of medications and unique medication identifiers than those depicted in FIG. 1.

Medication 100 may be any type of single dose of medication (or less than single dose, such as when a dose requires two or more pills, etc.) like a pill, on which a unique medication identifier may be placed. The term “Medication” includes prescription medication, over-the-counter medication, and any other form of consumable (e.g. nutritional supplements, vitamins, etc.) that is in the form of a tablet, pill or capsule. Medication 100 may be in any form, such as a capsule, caplet, a tablet, etc. Medication 100 may include one or more treatments (e.g. surface treatments, such as a coating on which unique medication identifier 110 may be placed, a flat area on which the unique medication identifier 110 may be placed, etc.) to facilitate the placement, integrity, scanning, etc. of the unique medication identifier 110. For example, and not limitation, providing a flat surface for the unique medication identifier 110 may allow the unique medication identifier 110 to be more easily scanned than when the medication 100 does not have the flat surface. As further example, and not limitation, the surface treatment may be included to ensure that the medication 100 is not negatively affected by the unique medication identifier 110 and/or that unique medication identifier 110 functions properly (e.g. surface treatment 105 may be additional material on a capsule where the unique medication identifier 110 is etched so that the capsule does not dissolve quicker, burst easier at the location of the unique medication identifier 110, may include a flat surface for the identifier or a sticker containing the identifier etc.). The unique medication identifiers 110 shown in FIG. 1 include quick response codes, or QR code and bar codes, but unique medication identifier 110 may be in many other forms that may be scanned by a scanning device to and used to identify medication 100, such as a matrix barcode, a label or sticker containing a code, a string of alphanumeric characters, microdot, taggant, micro taggant, molecular bar code, etc.

The unique medication identifier 110 may be applied to the medication in a variety of ways and using a variety of types of equipment. For instance, the unique medication identifier 110 may be etched to the surface of the medication 100, placed on a sticker or label (e.g. one that dissolves, is non-toxic, etc.), may be applied with inks, paints or other surface coatings, etc. many options for mechanically applying the unique medication identifier 110 directly to the medication 100 (rather than applying a sticker or other piece of material containing the unique medication identifier) exist and are known in the art (e.g. laser etching, printing, embossing, debossing, engraving, press marking, roll marking, dot open peen marking, stylus marking, stamps, stickers, overlays, scribes, microdot, taggant, etc.).

FIG. 1 depicts three medication identifiers 110, but the systems and/or methods described herein may be used in connection with one unique medication identifier 110 on a medication 100, multiple unique medication identifiers 110 (i.e., 2 unique medication identifiers, three unique medication identifiers, etc.) on a medication 100, a unique combination of non-unique identifiers that uniquely identifies the medication 100, etc. Additionally, or alternatively, a non-unique identification identifier (e.g. a barcode, any of the identifiers used as a unique medication identifier, a digit, a color, a stripe, etc.) may be included on the medication in addition to the unique medication identifier. Additional identifiers (i.e. other than the unique medication identifier 110, whether unique or non-unique) on the medication may allow further confirmation that a medication 100 is authentic, such as by interrogating the database described in further detail herein to determine whether the additional identifier is associated with the unique medication identifier 110 and, if not, allowing a determination that the medication 100 is counterfeit.

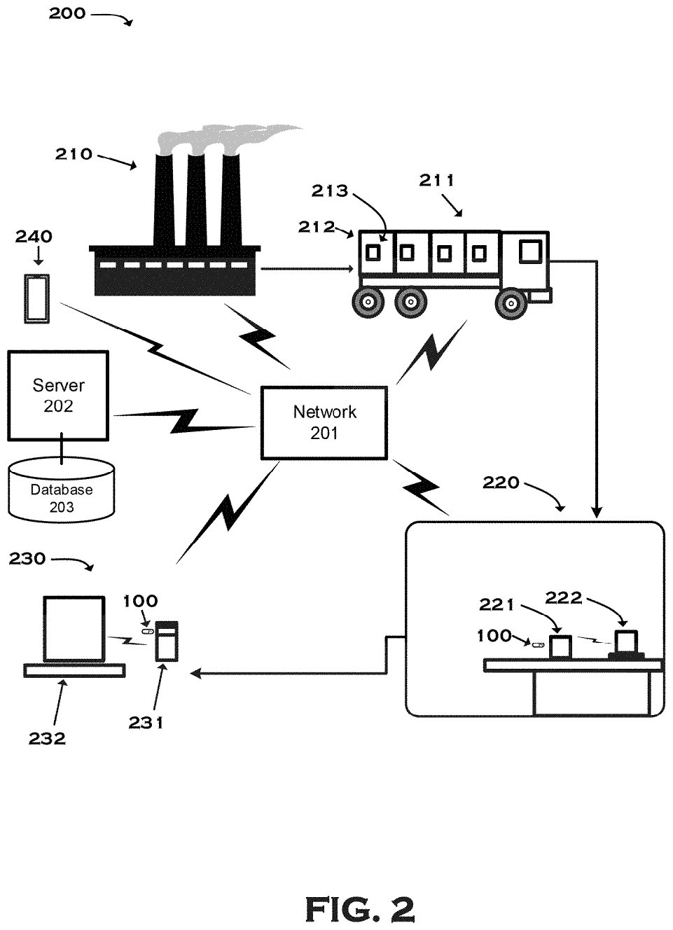

FIG. 2 illustrates a diagram of an example environment in which the systems and/or methods described herein may be implemented. As shown in FIG. 2, environment 200 may include a network 201, server device 202, manufacturing facility 210, distribution vehicle 211, dispensary 220, a scanning device environment 230, and user device 240. The dispensaries, servers, networks, distribution vehicles, scanning devices and/or other components shown in FIG. 2 are provided for explanatory purposes only, and the disclosure herein is not intended to be limited to what is reflected in FIG. 2. There may additional components, fewer components and/or differently arranged components than what is shown in FIG. 2. Also, in other implementations, one or more of the components of FIG. 2 may perform the function of one or more other components of FIG. 2. For example, and not limitation, the scanning devices described herein may perform the functions of the user devices described herein, the dispensary may input manufacturing information to server 202 via network 201, etc. FIG. 2 is a limited environment that is provided for explanatory purposes only; the systems and methods described herein may be applied on a large scale in environments that are regional, national, and/or global in nature.

Network 201 may include one or more wired and/or wireless networks via which the servers, scanning devices, user devices, etc., described herein, may communicate. For example, network 201 may include a wide area network (WAN) a metropolitan network (MAN), a telephone network (e.g. the Public Switched Telephone (PSTN)), an ad hoc network, an intranet, the Internet, a fiber optic-based network, and/or a combination of these or other types of networks. Additionally, or alternatively, network 201 may include a cellular network, a public land mobile network (PLMN), a second generation (2G) network, a third generation (3G) network, a fourth generation (4G) network (e.g., a long term evolution {LTE) network), a fifth generation (5G) network, and/or any other medium via which the server devices, user devices, scanning devices and/or other components described herein may communicate.

Server 202 may be any computation and communication device configured to gather, process, search, store, and/or provide information in a manner described herein. The database described herein may be hosted/stored on memory associated with server 202. Server 202 may be configured to communicate via network 201. Server 202 may provide a website and/or application that can be displayed, downloaded, and/or installed on the user devices, scanning devices and any other devices described herein to permit the transfer (e.g. uploading to server 202 or downloading from server 202) of the information associated with medication described herein and to further perform operations and/or display information as described herein. Server 202 may also, or alternatively, be configured to act as a web server or other type of server that hosts one or more websites and/or applications that may be accessed by the users of systems and/or methods described herein. Server 202 may include a collection of components typically found on server devices, such as a bus, processors, a memory, a read-only memory (“ROM”), a storage device, an input device, an output device, and/or a communication interface, etc.

Server 202 may perform certain operations in response to one or more processors executing software instructions contained in a computer-readable medium, such the memory of server 202. Further, server 202 may include on or more database 203 (hereinafter referred to as “database 203”), described herein, on the memory of server. Additionally, or alternatively, the software instructions and/or database 203 described herein may be read into memory from another computer-readable medium, such as a storage device, or from another device that communicates with server 202 via network 201 (e.g. another server device). The software instructions may cause the processing unit to perform processes described herein.

Database 203 may include one or more devices that store information received by and/or from server 202. For example, database 203 may store copies of an application that are supported by and/or compatible with different types of scanners (e.g. scanner 221, 231, etc.) user devices (user devices 222, 232, etc.) other servers, etc. Database 203 may also store a website (e.g., the code associated therewith) that scanners and/or user devices or any other server may access to send and/or receive the information associated with medication 100 and/or other information described herein. Database 203 may store one or more user profiles, such as profiles associated with manufacturer 210, those in distribution 211, dispensaries 220, patients, doctors, which user profiles may be associated with medication 100 in database 203. Database 203 may also, or alternatively, store the content described herein and the data and/or metadata associated therewith. Database 203 may store any in formation and/or data transmitted within environment 200.

Database 203 may store the information associated with medication described herein such that the information described herein may be provided in response to a query containing the unique medication identifier, the identity of the patient, or other information that may be used to query the database for related information. Additionally, or alternatively, database 203 may store the other information described herein, such as information associated with individuals, physicians, law enforcement, etc. Database 203 may store the information described herein in profiles to maintain the association between related information. For example, and not limitation, database 203 may include a medication profile for medication 100. The medication profile may include the unique identifier for medication 100 as well as all information associated with medication 100. As a result, when a user device queries database 203 for information associated with medication 100 using unique medication identifier 110 and the biometric information obtained by the user device, the medication profile in database 203 for medication 100 is identified when the unique medication identifier from the user device and the biometric information is matched to the unique medication identifier and biometric information in the medication profile for medication 100. Similarly, other entities used in connection with systems and methods described herein may have profiles. For example, an individual may have a profile containing, for example, allergy information for the individual, the fact that the individual may not receive opioids, a picture of the individual, etc. This profile may be searched (i.e. by the individuals name, by a unique identifier for the individual, etc.), such as by a pharmacist (i.e. server 202 may execute instructions to automatically search database 203, etc.) when filling the individual's prescription. The pharmacist may input the prescription in known ways, and this information may be provided to database to discover the individual's profile in the database 203. Server 202 may identify in database 203 the individual's profile, which may include the information associated with the individual. Server may also notify pharmacist (in response to, for instance, uploading prescription information, scanning medication 100, etc.) that the individual cannot be dispensed the medication (i.e. the individual is allergic, is not allowed by law to have the medication, etc.) based on the information in individual's profile. A person of ordinary skill in the art understands that multiple profiles, queries, etc. are taught by the systems and methods provided herein such that the information stored in the database ensures the safe and legal administration of medication.

Database 203 and server 202 may be controlled by manufacturer 210. Additionally, or alternatively, server 202 and/or database may be owned and/or controlled by a third party, a governmental entity charged with overseeing the dispensing of medication (e.g. the FDA, etc.), etc. There may be a single server 202 and/or database 203 or multiple servers 202 and/or databases 203 that function together in connection with the systems and methods described herein.

Server 202 may execute software instructions to allow the devices and components described herein (such as those associated with dispensary 220 and/or manufacturing facility 210 user device 240, etc.) to upload the information associated with medication described herein and to store the information associated with the medication in the database described herein. Additionally, or alternatively, server 202 may execute software instructions that allow a user, such that user device 231 associated with scanning device environment 230 (or user device 240 or user device 222) to query the database 203 associated with server 202 for the information associated with medication that corresponds to a unique medication identifier of a medication, such as medication 100, or to upload information to database 203.

Manufacturing facility 210 may be any facility associated with manufacturing the medication 100 described herein. Manufacturing facility 210 may manufacture the medication described herein, such as medication 100, and may apply unique medication identifier 110 to the medication in the ways described herein. Manufacturing facility 210 may also communicate with server 202 via network 201 (e.g. such as with a server or a user device associated with manufacturing facility) to provide information associated with medication to server 202, for instance, populate the database with information associated with medication 100 (e.g. the unique medication identifier for medication 100, type of medication, lot number, batch number, date of manufacture, expiration date, legal restrictions on the medication, and other information known to manufacturing facility 210, including shipping, packaging (which pallet, which box, which package, which bottle, etc.) and distribution information). Initially, manufacturing facility 210 may communicate with server device 202 to obtain the unique medication identifier 110 to be applied to a medication 100 and/or to provide the unique medication identifier (such as a “serialized ” alphanumeric string, the numbers of a barcode, etc.) to the database so that it can be associated with the medication 100. Manufacturing facility 210 may have additional equipment and/or processes that may be used in association with the systems and/or methods described herein. For example, manufacturing facility 210 may have quality control that may, in addition to other tasks, analyze the medication 100 to ensure that the unique medication identifier can be read by a scanning device (such as scanning devices 221 and 231), to ensure that applying the unique medication identifier did not affect the performance of the medication, etc.

Distribution 211 may be any type of distribution channel that is used in the medication industry, such as tractor trailer, intermodal containers on ships, airline shipments, railway shipments, and/or a combination of the foregoing. Distribution 211 represents the channels of trade between the manufacturing facility 210 and dispensary 220 and includes all warehouses, distributors, and other points along the distribution channel and all associated transportation. Distribution 211 may include a scanner and may communicate with server 202 to provide information associated with the medication (provide shipping information, storage information, etc.).

Typically, when medication 100 is in distribution 211, medication 100 resides in packaging 212, by way of example and not limitation, boxes, bottles, cartons, pallets, containers, and the like, so medication 100 cannot be individually scanned without removing medication 100 from packaging 212. Therefore, the packaging 212 may include an identifier 213, which may be a barcode (2D, 3D, etc.) and/or any of the other identifiers referenced herein in connection with unique medication identifier 110. All medication 100 in packaging 212 may be associated with identifier 213 in database so that, when identifier 213 is scanned, in formation associated with packaging (i.e. location, date, shipping method, etc.) is uploaded to database 203 (such as via network 201) in the ways provided herein. As medication 100 makes its way through the supply chain, packaging 212 may be opened to distribute the medication 100. For instance, packaging 212 may initially correspond to palletized cargo, and there may be an identifier 213 on the palletized cargo. All medication 100 in the palletized cargo may be associated with the identifier 213 for the palletized cargo in database 203, such as by manufacturer 210 providing this information to database 203. When the palletized cargo is divided into, for instance, boxed cargo, the boxed cargo may include a second identifier 213. The second identifier 213 may be associated in database 203 with all medication 100 in the boxed cargo, as this information would be known by manufacturer 210. The boxed cargo may be scanned by another in distribution 211 and/or by dispensary 220, such as when the boxed cargo arrives. Medication 100 may be packaged in many different ways, and distribution 211 may require additional identifiers 213 (three identifiers, four, five, etc.) based upon how medication is packaged. Regardless of the number of identifiers 213/packages 212, manufacturer 210 may associate in database 203 each medication 100 with all identifiers 213 in all packages 212 in which medication 100 is packaged so that medication 100 can be tracked throughout distribution 211 all the way to dispensary 220 (and even at dispensary 211 if, for instance, medication is in several layers of packaging at dispensary, moves around in dispensary 220, etc.). This may be accomplished by scanning identifier 213 at every point in distribution 211 so that medication 100 may be tracked from manufacturer 210 to dispensary 220. Tracking medication 100 at every point until it is consumed may further limit and/or prevent counterfeit medication from entering the supply chain, may allow authorities to determine which medications 100 were included in a problematic shipment (e.g., a stolen shipment, in which case database 203 may be updated to reflect this fact; a shipment in which medication was ruined due to temperature, etc.).

Dispensary 220 may be any facility at which medication 100 is provided to a consumer of medication 100. For example, and not limitation, dispensary 220 may be a pharmacy, a hospital, an assisted living facility, a nursing home, a clinic, etc. Dispensary 220 may include a scanning device 221, a user device 222 and medication, such as medication 100, which is dispensed to a consumer of medication. Prior to dispensing medication 100 to a consumer of medication, dispensary 220 (someone associated with dispensary, like a pharmacist, treating nurse, etc.) may scan medication 100 with scanning device 221 to determine the information associated with medication 100 that was uploaded to server 202 and resides in the database, such as the information uploaded to the database by manufacturing facility 210. Dispensary 220 may obtain the information associated with the medication 100 using scanning device 221 to communicate with server 202 via network 201 to request the information associated with medication 100 by a query including the unique medication identifier (or a serialized version thereof) of medication 100. Additionally or alternatively, scanning device 221 may communicate with user device 222 via network 201 and/or another network to provide the unique medication identifier for medication 100 to user device 222, and user device 222 may communicate with server 202 via network 201 to obtain information associated with medication 100. Additionally or alternatively, dispensary 220 may communicate with server 202 to identify a patient seeking medication 100. In response, server 202 may provide information in the database for the patient seeking medication, such as at least one prescription (outstanding or pending) associated with the patient, the patient's criminal history (e.g. convictions for drug-related offenses, etc.), physical health history, mental health history, or other information that may be relevant to dispensing the medication 100. In response to this information, an alert is generated for the at least one prescription for the corresponding patient. Additionally or alternatively, dispensary 220 may refuse to fill a prescription, alert authorities, alert a treating physician, etc. Additionally, or alternatively, server 202 may execute instructions to determine that a prescription should not be filled (i.e. may notify the pharmacy that the prescription should not be filled, may notify authorities of the fact that a patient is receiving additional medication, etc.). Additionally or alternatively, server 202 may execute instructions to determine that a dose of medication is counterfeit or weaponized, etc.

Scanning device 221 and/or user device 222 may provide information associated with the medication to server 202 via network 201 to further populate the database described herein. For instance, a user may operate scanning device 221 and/or user device 222 to provide the prescription information (the number of doses, the date the prescription was filled, the medical professional associated with the prescription, the patient to whom medication was prescribed, etc.) the name of the consumer of the medication (e.g. the patient, a guardian for the patient, etc.), the pharmacists or other professional filling the prescription, the date the prescription was filled, etc. to the database. This information may become associated with the medication 100 as additional information associated with medication in the database, which may be provided in response to future queries to the database for information associated with medication 100 by law enforcement or others.

In one example embodiment, a pharmacist at dispensary 220 may receive a prescription from a user for medication, such as medication 100, and begin filling the prescription by obtaining the number of doses needed to fill the prescription. Medication 100 (i.e. each dose of medication in the prescription) may by scanned by scanning device 221, and dispensary 220 may communicate (via scanning device 221 or user device 222) each of the unique medication identifiers 110, for each medication 100, to server 202 via network 201 to populate database 203 with this information, to request existing information associated with the medication 100 (each of the actual pills) being dispensed to the patient (i.e. to confirm that the medication is the correct medication to fill the prescription, is not subject to a recall, etc.). Alternatively, server 202 and/or database 203 may execute instructions to determine whether the medication 100 may be provided to the consumer (i.e. automatically check for recalls, drug interactions, criminal history, etc. Dispensary 220 may then populate the database by providing the information associated with medication 100 for each dose in the prescription (i.e. all prescription information and other information known by dispensary 220 and not yet included in the database 203, such as the name of the patient, the identity of the treating physician, the date the prescription was filled, etc.). This populates the database 203 with the information associated with medication 100 used to fill the prescription. Dispensary 220 may have additional equipment that may assist with the systems and/or methods described herein. For instance dispensary 220 (and any other locations in environment 200, such as manufacturing facility 210) may include a hopper or similar equipment that may align medication so that each dose of medication 100 may be quickly scanned (i.e. all doses of medication in a prescription, etc.), and the unique medication identifiers 110 may be used to query/populate the database efficiently for each medication 100. In other settings, by way of example and not limitation, when dispensary 220 corresponds to a hospital where individual doses of medication 100 are provided to a patient at the point of care, the dispensary 220 may scan or otherwise input data for the medication 100 and provide patient information to the database 203 (such as by scanning an identifier associated with patient) to confirm that the right patient is receiving the right medication at the right time, that has not been recalled, is not counterfeit, is not going to cause a drug interaction, etc.

The scanning device 221 may be any sort of device capable of interrogating medication 100 to read the unique medication identifier. For example, the scanning device 221 may be a barcode reader, an electro-optical device, a camera, smartphone, etc. Scanning device 221 may communicate with user device 222 and/or server 202 to transmit the unique medication identifier to identify, authenticate and/or provide the location of the medication. User device 222 may be a server device, like server 202, or similar device (e.g. a computer, a laptop, a smartphone, a tablet, etc.) that may communicate with server 202 via network to transmit information as described herein and to display such information to someone associated with the user device 222 (e.g. a pharmacist, law enforcement, the patient, etc.). The user device 222 can be equipped with biometric sensors for identifying the user accurately and to verify an association with a medication. Biometric sensors include but are not limited to fingerprint sensors, finger vein, iris sensors, facial sensors, etc. Scanning device 221 may be the same device and/or a separate device from user device 222 (e.g., scanning device 221 may be a computer device, a smartphone, or other handheld computer or reader that includes the ability to identify and interrogate the unique medication identifier as described herein).

Scanning device environment 230 may be any environment whereby medication 100 may be scanned by scanning device 231 to obtain information associated with the medication 100 from server 202 (i.e. stored on the database associated with server 202) via network 201. In the event that medication 100 is consumed at dispensary 220 (such as when, for instance, dispensary 220 corresponds to a hospital) scanning device environment 230 may not exist because medication has been consumed. When medication 100 is not consumed at dispensary 220 (such as, for example, when dispensary 220 corresponds to a pharmacy), scanning device environment 230 may be any environment in which medication 100 may be scanned after being dispensed. For example, and not limitation, scanning device environment 230 may be associated with the patient to whom the medication was prescribed (e.g. to determine whether medication 100 has expired, to determine whether medication 100 was recalled, to identify the medication and proper dosage, to determine when to take the medication 100 based upon prescription instructions associated with the medication 100 that are uploaded by a physician or other healthcare professional and associated with medication 100 by dispensary, pharmacy, etc.), law enforcement (e.g. to determine whether the individual in possession of medication 100 is the patient to whom the medication 100 was prescribed, to determine whether medication 100 is counterfeit or authentic, whether the manufacturer failed to properly update database, etc.) a medical professional or a first responder (e.g. to identify the medication, the dosage, etc.). Scanning device environment 230 may include a scanning device 231 (which may be the same as, or similar to, scanning device 221) and/or user device 232 (which may be the same as, or similar to, user device 222). Scanning device 231 may scan medication 100 to determine the unique medication identifier of medication 100. The unique medication identifier of medication 100 may be used to query the database 203 by communicating with server 202 via network 201 to obtain the information associated with medication 100. Scanning device 231 may communicate with server 202 to obtain the information associated with medication 100. Additionally, or alternatively, scanning device 231 may communicate with user device 232 via network 201 and/or another network to provide user device 232 with the unique medication identifier for medication 100 to allow user device 232 to query server 202 for information associated with medication 100.

User device 240 may be another user device, like user devices 222 and 232, that may communicate with server 202 via network 201 download information associated with medication and/or to upload information, such as information associated with medication or information associated with patients, information associated with laws, etc. User device 240 may be associated with any person or entity having access to database 203. In one example, user device 240 is associated with manufacturing facility 210, and manufacturing facility 210 populates database 203 with information associated with the medication. In another example, user device 240 is associated with law enforcement, and law enforcement populates the database with information associated with the medication (e.g. the medication has been weaponized, banned, is subject to age verification when distributed, etc.) or information associated with patients (e.g. the identity of individuals to whom prescriptions for opioids cannot be dispersed, etc.). In yet another example, user device 240 is associated with a medical practice, which populates the database with information associated with patients (i.e. when to take medication, how much, allergies, etc.) The information provided by user device 240 to database may be used in a variety of ways to make a variety of decisions, and server device 202 (or any of the user devices) may combine the information in the database 203 with the information uploaded to the database 203 by user device 240 to evaluate in order to make decisions. For example, law enforcement may upload a list of individuals to whom opioids may not be dispersed to database 203. Dispensary 220 may upload to database 203 the identity of a patient to whom medication 100, in this example an opioid, was prescribed. Server 202 may execute instructions as described herein to determine that the patient is on the list of individuals uploaded by law enforcement, and the server 202 may communicate with user device 222 of dispensary 220 to instruct dispensary 220 not to dispense medication 100 to the patient. In another example, a doctor may use user device 240 to upload allergy information (i.e. a list of medications to which patient is allergic) of a patient to database 203. Dispensary 220 may attempt to fill a prescription for medication 100 (medication to which patient is allergic in this example), and server 202 may instruct dispensary via user device 222 not to fill the prescription for medication 100 to patient as a result of the allergy information

The information associated with medication that is provided in response to the queries described herein may vary depending upon, for instance, what information has been populated in the database as well as permissions of the device associated with the query. For instance, if the device is associated with law enforcement, the information associated with medication may be most and/or all of the information associated with the medication. If the device is associated with an unknown individual, or an individual associated with profile for which limited access is granted, then the information associated with medication provided in response to the query may be limited, such as limited to the type of medication, dosage, whether it has been recalled, has expired, etc. Permissions may be based upon software instructions on server 202. Additionally, or alternatively, server 202 may execute software instructions to determine permissions associated with read/write access to the database that, for instance, may restrict the ability of many devices to upload information associated with medication to the database (i.e. only devices associated with manufacturers, dispensaries, and certain others may upload information associated with medication).

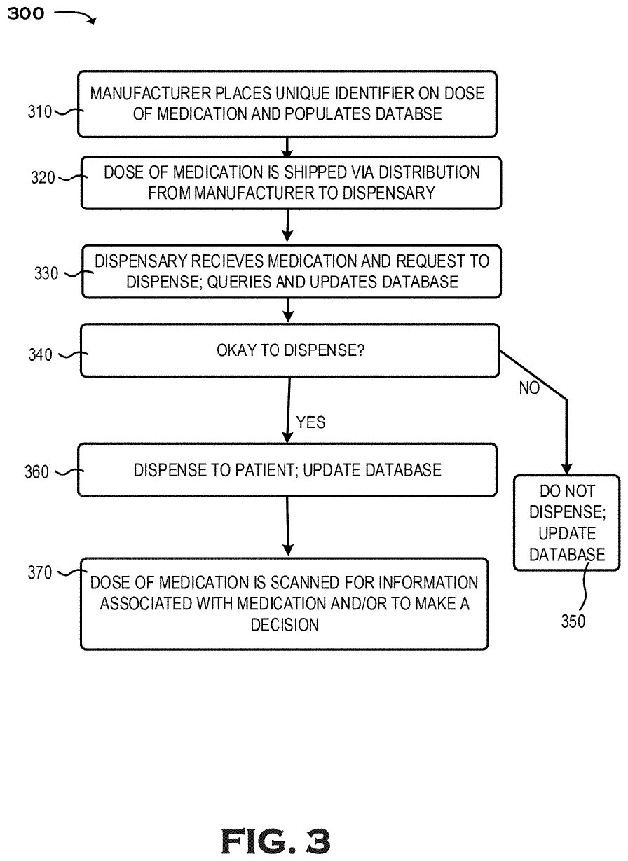

FIG. 3 is a flow chart of an example process 300 that may be used to provide information according to an implementation described herein. According to the process, manufacturer 210 may manufacture a medication 100 and may place a unique medication identifier 110 on the medication 100. (BLOCK 310). In one embodiment, a manufacturer 210 queries the database 203 to provide via the network 201 a randomized, non-sequential, non-duplicating unique medication identifier. The medication 100 and unique medication identifier 110 may include any of the types described herein. The manufacturer 210 may populate a database (such as stored on server 202) to provide information associated with the medication (i.e. the type of medication, the dose of medication, the ingredients, the provider of ingredients, the identity of the medication, the manufacturer, manufacture date, expiration date, supplier information, shipping information, packaging information and identifiers, etc.). The manufacturer 210 may also provide the unique medication identifier to the database (i.e. by providing a serialized version of the unique medication identifier, etc.). The unique medication identifier 110 may be linked to the information associated with the medication 100 in the database such that, when the unique medication identifier is used to query the database, the database may provide the information associated with the medication.

The medication 100 may be shipped from the manufacturer to a dispensary 220. (BLOCK 320). Shipping the medication to the dispensary includes any route a medication may take through a supply chain, including transportation on trucks, planes, trains, vessels, etc., as well as storage and distribution. Typically, the medication 100 is shipped in packaging 212 including but not limited to containers, boxes, bottles, pallets, etc. which prevent scanning the individual dose of medication 100. However, the packaging 212 may include identifiers 213 which may be scanned during the distribution process to provide database 203 with information on the location of the medication 100, dates on which medication 100 arrives at a point in the supply chain, etc.

Dispensary 220 may receive the medication 100 from distribution 211 and may scan medication 100 or packaging for medication and update database 203 to reflect receipt of medication. (BLOCK 330) Additionally, or alternatively, distribution 211 may scan identifier 213 on packaging 212 of medication 100 and update database 203 in the ways described herein to reflect the fact that medication 100 has been delivered to dispensary 220. Dispensary 220 may receive a request to dispense the medication 100, such as a prescription (in the event dispensary is a pharmacy) an order to dispense to a patient in a hospital, etc. Dispensary 220 may scan the medication 100 to query database 203 to obtain information associated with the medication 100. The information associated with the medication 100 may include, for instance, the type of medication, the dose, whether the medication has expired, is subject to a recall, is authentic or counterfeit, is weaponized, etc. The information associated with the medication 100 may allow the dispensary 220 to determine that the medication 100 may be dispensed or may not be dispensed. (BLOCK 340). As a result of this determination, dispensary 220 may dispense the medication (BLOCK 360) or not dispense the medication (BLOCK 350). Dispensary 220 may update the database 203 to reflect whether the medication was dispensed, not dispensed and/or the reasons therefore. (BLOCK 350/360).

Dispensary 220 may also, or alternatively, query the database 203 to obtain information associated with the patient to whom the medication 100 may be dispensed. (BLOCK 330) The information associated with the patient may include, for instance, the identity of the patient (name, age, address, etc., which may be used to confirm the identity of the patient), whether the patient has been identified in the database as restricted individual (e.g. an individual with a criminal record for illegally using and/or distributing opioids, an individual who may not receive medication, such as at a hospital, because the last dose was given too recently, is taking another medication that would cause a dangerous reaction if take with medication 100, has recently had a prescription for same or same class of medication filled and is not eligible to receive that medication or medication type, etc.). The restricted nature of the individual may be included in the database 203 as a result of input from a user and/or determined by software instructions executed by the server 202. The dispensary 220 may analyze the information associated with the patient to determine whether to dispense the medication (BLOCK 340). As a result of this determination, dispensary 220 may dispense the medication (BLOCK 360) or not dispense the medication (BLOCK 350). Dispensary 220 may update the database 203 to reflect whether the medication was dispensed, not dispensed and/or the reasons therefore. (BLOCK 350/360).

If the medication 100 is dispensed but not consumed immediately thereafter (such as would be the case when dispensary is a pharmacy, rather than being dispensed in a hospital room to a patient), medication 100 may be scanned at a later point in time. For example, and not limitation, law enforcement may scan medication 100 to query the database 203 to determine whether medication 100 is authentic or counterfeit, whether the medication was prescribed to an individual, etc. A consumer of the medication 100 may scan the medication 100 to query the database 203 to determine, for example, the identity of the medication (medication type, dose, manufacturer, etc.), whether the medication is subject to a recall or past the expiration date, etc. Based upon this information, a decision associated with the medication may be made, such as consuming the medication, not consuming the medication, arresting the possessor of the medication, impounding counterfeit medication, etc. (BLOCK 370) The patient may input information such as if the medication has been stolen, has caused a reaction or secondary (unintended) response, etc. A person of ordinary skill in the art will recognize that the information that may be stored in database 203 may be used to make a number of decisions, and that the disclosure herein is not limited to the example information, decisions, etc. recited in the examples provided herein.

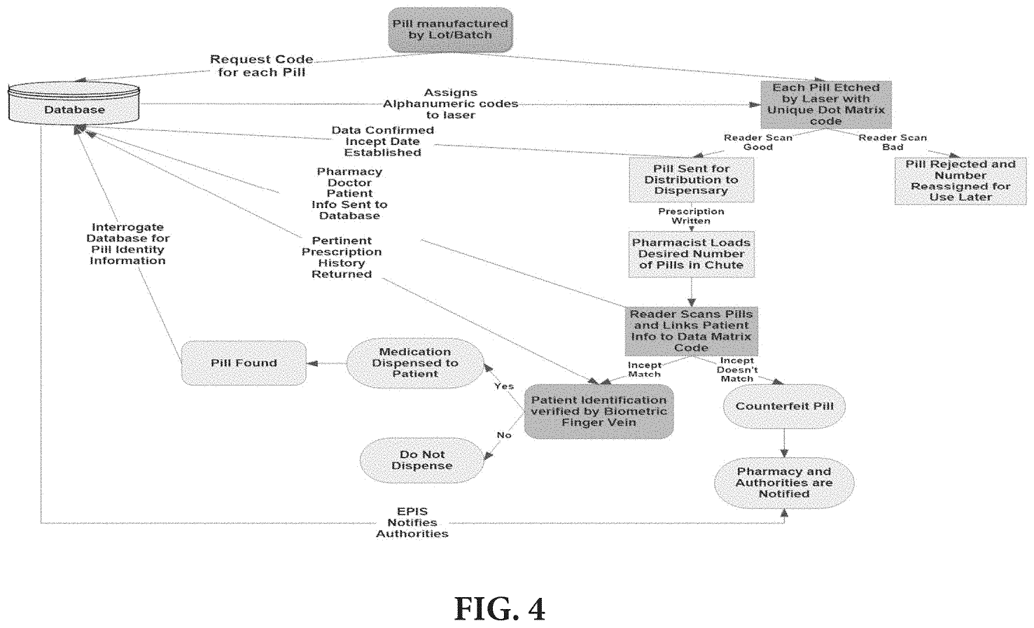

FIG. 4 is a schematic illustrating the process for marking a medication according to one embodiment of the present invention. In FIG. 4, a manufacturer requests a code for each unit of medication for medication that is manufactured in lots or batches. Each pill is etched by laser with a unique dot matrix code, and each code is scanned. Upon a finding that the code is not readable, the medication unit is rejected and the unique dot matrix code is reassigned for later use. Upon a finding that the unique dot matrix code is readable, the medication unit is sent to a dispensary for distribution and the database is updated with the unique dot matrix code for each medication unit and a date of incept for each medication unit. After a prescription is written for the medication unit, the medication unit identifier is scanned along with the medication unit identifiers of any other medication units in the prescription and pharmacy information, patient information, and doctor information is linked to the medication unit in the database. If an incept date does not match the date in the database, a determination that the medication unit is counterfeit is made and the pharmacy and relevant authorities are notified. If the incept date matches, a patient's biometric information such as biometric finger vein information is provided by the database and matched at the pharmacy. Upon a matching of the patient's biometric information in real-time or near real-time at the pharmacy, the medication unit is dispensed. If the patient's biometric information does not match, the medication unit is not dispensed. If a medication unit is later discovered, the database is operable to be queried to determine identifying information for the medication unit including but not limited to, the identity of the pharmacy, the doctor prescribing the medication unit, the patient who was prescribed the medication unit, the dosage, the date of prescription, the expiration date, etc.

The system and method of the present invention can be used for research purposes at the governmental, pharmaceutical, medical, etc. levels to discover and link key details to a medication such as if the medication is not safe for use among a certain age group, ethnicity, gender, etc. The bulk of medications that hit that market are not tested on a female population or lack a representative female population, which leads to a delay in the discovery of sex-related issues until after a widespread use has begun (FDA Research, Policy, and Workshops on Women in Clinical Trials, last accessed Sep. 13, 2019 at https://www.fda.gov/science-research/womens-health-research/fda-research-policy-and-workshops-women-clinical-trials). The disclosed system can be especially useful in tracking the efficacy and safety of a new medication on the market and pinpoint issues at a much faster rate than the current method. For example, a physician or user may input to the database information pertaining to the medication such as side effects that may be found to be associated with certain user's traits such as age, gender, ethnicity, etc. Researchers would be able to access the pertinent information in the database associated with a medication to draw crucial ties between medications on the market and their users to help government agencies make well-informed decisions.

The United States is at threat by China's control and knowledge of drug manufacturing. China is estimated to produce 80% of the ingredients that go into drugs and produce around 70% of the drugs that enter the U.S. market. China claims to be able to weaponize their role in drug making if economic tensions between the U.S. and China continue by halting their production and export of drugs into the U.S. The U.S. needs to be ready for such an attack by having the necessary data associated with drug usage in the U.S. so that transitioning the manufacturing into the U.S. can be streamlined and effective. The disclosed system for tracking medication can provide the necessary data for the government to take on this threat as well as others. For example, if a natural disaster was approaching a state, the government can predict how much of each medication will be needed for the users in that area as well as which drugs will be needed over a certain period of time to prepare an effective response to the disaster.

Continuous monitoring of the database is possible for determining the effectiveness, risk factors, weaponized medications and/or nutritional supplements and compliance of patients with their medications among other anticipated uses. In one embodiment, an application on a mobile computing device scans the unique medication identifier for a medication unit, and is operable to automatically remind the patient when a next dose or a refill is due. This is accomplished by the application creating calendar appointments on a calendar application on the mobile computing device within the application or through a third-party application such as APPLE's CALENDAR, GOOGLE's CALENDAR, or any other calendar or appointment system. In another embodiment, the application is operable to automatically schedule the placing of a refill order for the patient, such as through GOOGLE ASSISTANT, if prescription refills remain on the patient profile. The mobile application is preferably operable to provide one or more reminder notifications via the mobile computing device relating to the next dose, the refill, etc.

Strategic monitoring, and machine-learning and/or AI-assisted monitoring can highlight geographical areas that may be affected by a medication deviation such as contamination, product tampering, etc., supply chain issues or problems, linking medication effects to a certain group of people, identifying unusual prescription fulfillment patterns compared with other demographics, and then automatically notify affected and/or potentially affected patients, government oversight agencies, law enforcement, hospitals, or other medical professionals or service providers, as appropriate, depending upon the data and legal, medical, privacy, and other factors.

In another embodiment, the present invention is utilized in applications where prescriptions are shipped to patients. As goods and services are offered remotely through the Internet, online pharmacies are also becoming more popular, and provide for receiving medication, including controlled substances, through delivery such as mail or drone delivery. There are numerous issues with receiving controlled substances such as opioids through the mail, and some states accordingly still prohibit patients from receiving controlled substances in this way. For example, it is difficult to verify that the patient to whom the medication was prescribed actually received the medication. The present invention solves this problem by requiring a patient to present biometric identification to receive medication before possession of the medication is transferred to the patient. In one embodiment, this is accomplished by a delivery person requiring biometric verification before handing over a package including a controlled substance or other medication. Biometric verification is performed through a mobile electronic device carried by the delivery person in one embodiment. In one embodiment, this mobile electronic device includes one or more biometric sensors operable to read a fingerprint, one or more facial features, one or more features of an iris, one or more features of one or more veins including finger veins (e.g. finger vein recognition), palm veins (e.g. palm vein recognition), or any other biometric identifier, individually or in combination. The mobile electronic device is connected to the database of the present invention and is operable to authenticate a patient's identity as the person who was prescribed the medication. The biometric data is preferably matched against the database, and the delivery person simply receives a confirmation of the identity of the patient or a confirmation that the patient is authorized to receive the prescription. Alternatively, a caregiver or guardian is operable to have their biometric data associated with the prescription and the device receives biometric data from the caregiver or guardian to authorize release of the package including the prescription. In another embodiment, a patient (or caregiver or guardian) has a mobile electronic device which is operable to receive biometric data through one or more biometric sensors and the mobile electronic device of the delivery person is operable to receive confirmation from the mobile electronic device of the patient that the patient, caregiver, or guardian is authorized to receive the prescription. This confirmation is preferably received in real-time or near real-time immediately before or upon delivery. In yet another embodiment, the patient, caregiver, or guardian scans a code on a package including the prescription using the mobile electronic device of the patient and the patient, caregiver, or guardian is authorized to receive the package including the prescription by providing one or more biometric identifiers through the one or more biometric sensors simultaneously, in real-time, or in near real-time. In a drone delivery embodiment, biometric data of a patient, caregiver, or guardian is provided to the drone through the mobile electronic device or is captured by the drone directly. Preferably, the drone holds the prescription in a locked compartment which is unlocked upon the drone receiving the biometric information.

In another alternative, a patient, caregiver, or guardian, is required to scan a code on a container including the prescription or the medication units individually to confirm that the appropriate patient received the prescription within a certain time period of receiving the package including the prescription. Notably, this also provides a method for a patient to confirm that the prescription is legitimate, or to learn that a medication is counterfeit.

In a telemedicine embodiment, a telemedicine provider is required to receive biometric data of a patient, guardian, or caregiver that identifies the patient, guardian, or caregiver as authorized to participate in the telemedicine appointment before the telemedicine provider starts an appointment or provides assistance to the patient. In one embodiment, the biometric data of the patient, caregiver, or guardian provides access to the telemedicine provider through an application on the mobile electronic device upon the mobile electronic device receiving the biometric data, with no other data being required from the patient, caregiver, or guardian to start the appointment. The biometric data is preferably matched against the database, and the telemedicine provider receives a confirmation of the identity of the patient, guardian, or caregiver. Preferably, this information is provided by the patient, guardian, or caregiver in real-time or near real-time. Alternatively, a patient, guardian, or caregiver is required to provide biometric data so that the telemedicine provider can write a prescription for the patient or the patient, guardian, or caregiver can receive the prescription. Biometric data preferably includes facial recognition according to these embodiments of the present invention, but is also operable to include any other type of biometric data recited herein. In one embodiment, prescriptions of the patient are linked to the biometric data such that the telemedicine provider is operable to view past prescriptions of the patient, past compliance of the patient with prescriptions, and other pertinent data (in accordance with HIPAA).

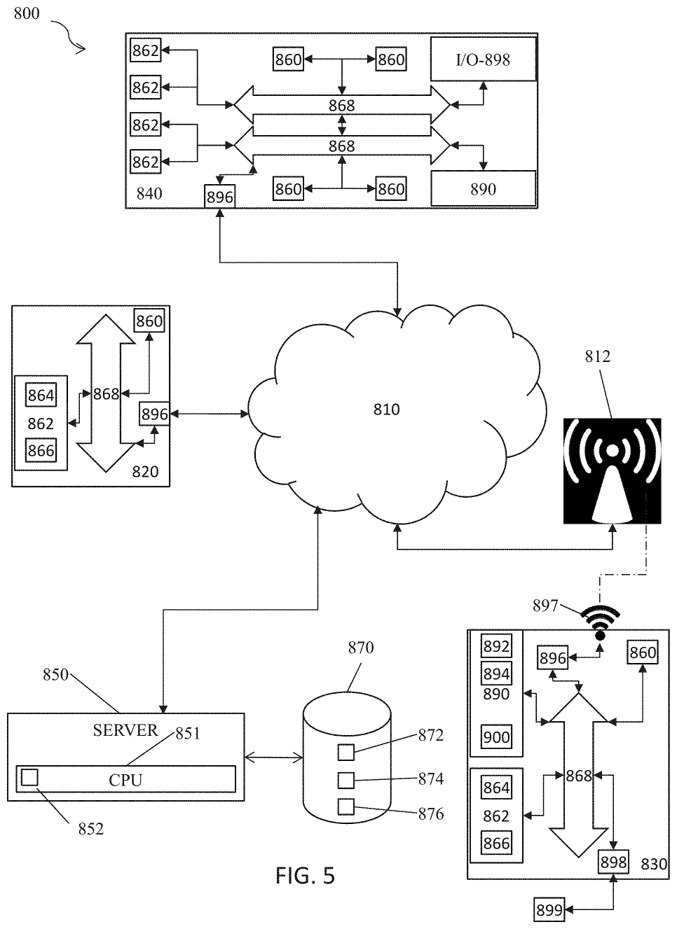

FIG. 5 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 may house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 may be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 may additionally include components such as a storage device 890 for storing the operating system 892 and one or more application programs 894, a network interface unit 896, and/or an input/output controller 898. Each of the components may be coupled to each other through at least one bus 868. The input/output controller 898 may receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 5, multiple processors 860 and/or multiple buses 868 may be used, as appropriate, along with multiple memories 862 of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function.

According to various embodiments, the computer system 800 may operate in a networked environment using logical connections to local and/or remote computing devices 820, 830, 840 through a network 810. A computing device 830 may connect to a network 810 through a network interface unit 896 connected to a bus 868. Computing devices may communicate communication media through wired networks, direct-wired connections or wirelessly, such as acoustic, RF, or infrared, through an antenna 897 in communication with the network antenna 812 and the network interface unit 896, which may include digital signal processing circuitry when necessary. The network interface unit 896 may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications, or other data embodying any one or more of the methodologies or functions described herein. The computer readable medium may include the memory 862, the processor 860, and/or the storage media 890 and may be a single medium or multiple media (e.g., a centralized or distributed computer system) that store the one or more sets of instructions 900. Non-transitory computer readable media includes all computer readable media, with the sole exception being a transitory, propagating signal per se. The instructions 900 may further be transmitted or received over the network 810 via the network interface unit 896 as communication media, which may include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 may not include all of the components shown in FIG. 5, may include other components that are not explicitly shown in FIG. 5, or may utilize an architecture completely different than that shown in FIG. 5. The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

The foregoing description provides illustration and description, but is not intended to be exhaustive or to limit the implementations to the precise form disclosed. Modifications and variations are possible in light of the above teachings or may be acquired from practice of the embodiments.

It will be apparent that systems and methods, as described above, may be implemented in many different forms of software, firmware, and hardware in the implementations illustrated in the figures. The actual software code or specialized hardware used to implement these systems and methods is not limiting of the implementations. Thus, the operation and behavior of the systems and methods were described without reference to the specific software code—it being understood that software and hardware can be designed to implement the systems and methods based on the description herein.

It should be emphasized that the terms “comprises” and “comprising”, when used in this specification, are taken to specify the presence of stated features, integers, steps or components but do not preclude the presence or addition of one or more other features, integers, steps, components or groups thereof.

Even though particular combinations of features are recited in the claims and/or disclosed in the specification, these combinations are not intended to limit the disclosure of the embodiments. In fact, many of these features may be combined in ways not specifically recited in the claims and/or disclosed in the specification. Although each dependent claim listed below may directly depend on only one other claim, the disclosure of the embodiments includes each dependent claim in combination with every other claim in the claim set.

No element, act, or instruction used in the present application should be construed as critical or essential to the implementations unless explicitly described as such. Also, as used herein, the article “a” is intended to include one or more items. Where only one item is intended, the term “one” or similar language is used. Further, the phrase “based on” is intended to mean “ based, at least in part, on” unless explicitly stated otherwise.

The invention claimed is:

1. A medication unit identifier for identifying a medication unit, comprising:
a unique medication unit identifier placed directly on a medication unit, placed within the medication unit, or attached to the medication unit;
wherein the unique medication unit identifier uniquely identifies the medication unit compared to all other medication units;
wherein the medication unit includes a single dose of a medication; and
wherein the unique medication unit identifier is associated with at least one biometric identifier operable to identify a user to whom the medication unit is prescribed.

2. The medication unit identifier of claim 1, wherein the unique medication unit identifier is a randomized identifier which is non-sequential relative to other unique medication unit identifiers for medication units within a prescription and is non-duplicated compared to all other unique medication unit identifiers.

3. The medication unit identifier of claim 1, wherein the at least one biometric identifier is operable to be captured and/or provided by at least one mobile computing device.

4. The medication unit identifier of claim 1, wherein the at least one biometric identifier includes a fingerprint, one or more facial features operable to be recognized via facial recognition software, one or more features of an iris, one or more features of one or more veins, or any other unique identifying feature of a user.

5. The medication unit identifier of claim 1, wherein the at least one biometric identifier is provided automatically to a scanning device or a device connected to the scanning device when the medication unit is scanned, photographed, or otherwise received as an input via the scanning device at a time of distribution or a time of consumption of a medication unit.

6. The medication unit identifier of claim 1, further comprising a container including the medication unit, wherein the container includes a unique container identifier, and wherein the unique medication unit identifier is linked to the unique container identifier, wherein the unique container identifier is linked to a pallet with a unique pallet identifier, and wherein the unique pallet identifier is linked to all of the unique container identifiers held within and associated unique medication unit identifiers.

7. The medication unit identifier of claim 1, wherein the medication unit includes a prescription medication, an over-the-counter medication, a vitamin, a nutraceutical, a botanical supplement, or a nutritional supplement.

8. The medication unit identifier of claim 1, wherein the unique medication unit identifier includes a microdot code, a taggant, a micro-taggant, and/or a molecular barcode.

9. The medication unit identifier of claim 1, further including a surface treatment, wherein the unique medication unit identifier is located proximate to the surface treatment.

10. The medication unit identifier of claim 1, wherein no other medication unit has an identical unique medication unit identifier for a predetermined period of time, wherein the predetermined period of time is based on medication shelf life and/or is based on a distribution expiry.

11. A system for uniquely identifying a medication unit comprising:
a unique medication unit identifier located directly on the medication unit, within the medication unit, or directly attached to the medication unit, the unique medication unit identifier distinguishing the medication unit from any other medication unit, including other medication units within a single prescription;
a database hosted on a server computer, the database being searchable to identify the medication unit, the database including the unique medication unit identifier and information associated with the medication unit on which the unique medication unit identifier is located; and
a device including a reader operable to read the unique medication unit identifier, wherein the device is operable to retrieve information associated with the medication unit from the database on the server computer upon the reader reading the unique medication unit identifier;
wherein the information associated with the medication unit includes at least one biometric identifier for an intended consumer of the medication unit.

12. The system of claim 11, wherein the device including the reader is operable to send a reminder relating to a next dosage date or a next dosage time for another medication unit within the single prescription.

13. The system of claim 11, wherein the at least one biometric identifier is provided automatically when the unique medication unit identifier is scanned, photographed, or otherwise received as an input via the device including the reader.

14. The system of claim 11, wherein the device including the reader or a device in network communication with the device including the reader communicates with the server computer via the network to provide information associated with a patient to the database, the information associated with the patient including an allergy status of the patient, a criminal history of the patient, or a prescription history of the patient.

15. The system of claim 11, wherein the device including the reader or a device in network communication with the device including the reader communicates with the server computer via the network to provide additional information associated with medication unit to the database, wherein the additional information associated with the medication unit includes:
an identity of a patient to whom the medication unit was prescribed;
shipping information for the medication unit;
an identity of a medical professional who prescribed the medication unit; and/or an identity of a pharmacy which filled the prescription for the medication unit.

16. The system of claim 11, wherein the information associated with the medication unit includes an expiration date of the medication unit, medications that should be avoided when taking the medication unit, or a recall status of the medication unit.

17. The system of claim 11, wherein the unique medication unit identifier includes a two-dimensional (2D) barcode, a three-dimensional (3D) barcode, a string of alphanumeric characters, a microdot code, a taggant, a micro-taggant, and/or a molecular barcode.

18. A system for uniquely identifying a medication unit comprising:
a unique medication unit identifier located on the medication unit, within the medication unit, or attached to the medication unit, the unique medication unit identifier distinguishing the medication unit from any other medication unit, including other medication units within a single prescription by the unique medication unit identifier not being identical to any other medication unit identifier; and
an application on a device including a reader operable to read the unique medication unit identifier, wherein the application is operable to retrieve information associated with the medication unit from a database on a server computer upon the reader reading the unique medication unit identifier;
wherein the device further includes at least one biometric reader, wherein the at least one biometric reader is operable to read a biometric identifier, and send information relating to the biometric identifier to the database on the server computer to be associated with the unique medication unit identifier.

19. The system of claim 18, wherein the biometric identifier includes a fingerprint, one or more facial features operable to be recognized via facial recognition software, one or more features of an iris, one or more features of one or more veins, or any other unique identifying feature of a user, and wherein the unique medication unit identifier includes a two-dimensional (2D) barcode, a three-dimensional (3D) barcode, a string of alphanumeric characters, a microdot code, a taggant, a micro-taggant, and/or a molecular barcode.

20. The system of claim 18, wherein the unique medication unit identifier is a randomized identifier which is non-sequential relative to other unique medication unit identifiers for medication units within a prescription and is non-duplicated compared to all other unique medication unit identifiers.
